(19) United States
(12) Patent Application Publication
(10) Pub. No.: US 2024/0424180 A1
(43) Pub. Date: Dec. 26, 2024

(54) PERITONEAL DIALYSIS CYCLER USING SORBENT

(71) Applicants: BAXTER INTERNATIONAL INC., Deerfield, IL (US); BAXTER HEALTHCARE SA, Glattpark (Opfikon) (CH)

(72) Inventors: Ramkumar JEYACHANDRAN, Bangalore (IN); Anders J. WELLINGS, Deerfield, IL (US)

(21) Appl. No.: 18/685,149

(22) PCT Filed: Oct. 4, 2022
(86) PCT No.: PCT/US2022/077516

§ 371 Date: Feb. 20, 2024

(30) Foreign Application Priority Data
Oct. 6, 2021 (IN) 202141045385

(51) Int. Cl. A61M 1/28

(52) U.S. Cl. CPC A61M 1/282; A61M 2205/7509

(57) ABSTRACT

A peritoneal dialysis (“PD”) machine includes a fresh/regenerated PD fluid pump, a used PD fluid pump, a sorbent cartridge located fluidically between the fresh/regenerated PD fluid pump and the used P fluid pump, a degassing tank located fluidically downstream from the sorbent cartridge, at least one valve located fluidically upstream of the degassing tank, and a control unit configured to control the fresh/regenerated PD fluid pump and the used PD fluid pump, the control unit further figured to operate the at least one valve to select whether fresh PD fluid or regenerated PD fluid is pumped by the fresh/regenerated PD fluid pump to the degassing tank.

### PRIORITY CLAIM

The present application claims priority to and the benefit of Indian Provisional Application No. 202141045385, filed on Oct. 6, 2021, the entire contents of which are hereby incorporated by reference and relied upon.

### BACKGROUND

The present disclosure relates generally to medical fluid treatments and in particular to dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the patient's peritoneal cavity, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

In any of the above modalities using an automated machine, the automated machine operates typically with a disposable set connected to a plurality of fresh PD fluid bags for a single treatment, e.g., storing a total treatment volume of perhaps twelve liters. Boxes of PD fluid bags for multiple treatments need to be stored in the patient's home, consuming space. The PD fluid bags can be heavy and cumbersome to transport from the home storage area to the APD machine for treatment, especially for older PD patients. Also, the use of a large volume of premade fresh PD fluid for each treatment, e.g., twelve liters, adds cost per treatment.

For each of the above reasons, it is desirable to provide an APD machine that reduces the amount or volume of premade fresh PD fluid needed per treatment.

### SUMMARY

Known automated peritoneal dialysis (“PD”) systems typically include a machine or cycler that accepts and actuates a pumping cassette having a hard part and a soft part that is deformable for performing pumping and valving operations. The hard part is attached to tubes that extend to various bags. The disposable cassette and associated tubes and bags can be cumbersome for a patient at home to load for treatment. The overall amount of disposable items may also lead to multiple setup procedures requiring input from the patient, which can expose room for error.

In a first main feature of the present disclosure, the PD system and associated methodology regenerates used PD fluid during treatment using a sorbent cartridge. The regeneration allows for a smaller number of PD fluid containers or bags to be used, lessening disposable waste, logistics regarding disposables and patient storage needed for disposable items. The regeneration also allows for less dialysis fluid volume to be stored for each treatment and reduces the amount or volume of premade fresh PD fluid needed per treatment. Reducing disposable waste, stored dialysis fluid volume and fresh PD fluid volume needed per treatment results in lower logistical costs (e.g., shipping and waste removal) and treatment cost (less premade fresh PD fluid needed).

The PD system in an embodiment includes four pumps. Three of the pumps are fluid pumps such as peristaltic pumps. The three fluid pumps may include a first, patient fill pump that pulls fresh PD fluid either from an initial PD fluid container or bag or from a degassing tank that stores sorbent regenerated PD fluid and pushes the fresh or regenerated PD fluid to the patient. The second fluid pump may be a used PD fluid pump that pulls used PD fluid from that patient, through a dialyzer, and pushes the used PD fluid to the sorbent cartridge for regeneration or to a drain if treatment has been completed. The third fluid pump is a concentrate pump that pulls electrolyte and glucose concentrates from containers of same and pushes the electrolyte and glucose concentrates into the degassing tank for mixing with regenerated PD fluid from the sorbent cartridge. The added electrolyte and glucose make up for electrolyte and glucose consumed during the patient dwell phase and removed by the sorbent cartridge.

The fourth pump is in one embodiment an air pump that removes air from the degassing tank when needed and delivers air to drain, e.g., a drain container or house drain such as a toilet or bathtub. The forth or air pump may be a vacuum pump.

The PD system in an embodiment includes multiple valves, e.g., seven valves. In an embodiment, multiple ones of the valves are three-way valves. The three-way valves are electrically actuated solenoid valves in one embodiment, each including a normally open (“NO”) port, a normally closed (“NC”) port and a common port. When energy is applied to the three-way valves, the ports switch states such that the NO port closes (restricts flow to the common port) and the NC port opens (allows flow to the common port). The NO port may be the more commonly used port to minimize the amount of time that the three-way valves need to be energized. The valves may alternatively or additionally include two-way valves, which again may be electrically actuated solenoid valves that are energized open so as to be fail safe.

The valves may include a first valve, which is a three-way source valve that determines where PD fluid for a patient fill is sourced. The source valve may be normally open to the sorbent cartridge to receive regenerated PD fluid for a patient fill (more patient fills use regenerated PD fluid so less energy used by making the NO port the regenerated port). The source valve is normally closed to an initial, e.g., two-liter container or bag of fresh PD fluid. A second valve may be a three-way valve located downstream from the first valve and be used primarily during patient fills for treatment and for creating a disinfection loop during a disinfection sequence at the end of treatment. A third valve may be a two-way patient fill valve, which is a two-way valve normally closed to the patient. The patient fill valve is energized open during a patient fill.

A fourth valve may be a two-way patient drain valve, which is a two-way valve that is also normally closed to the patient. The patient drain valve is energized open during a patient drain. Providing separate fill and drain valves allows fresh and used PD fluid to be separated and maintained in different permanent, reusable lines. In an embodiment, only a flexible patient line leading from the cycler to the patient receives both fresh and used PD fluid. A fifth valve may be a three-way valve located downstream from the two-way patient drain valve. The fifth valve determines where the used PD fluid from the patient is delivered, e.g., to the sorbent cartridge for regeneration for another patient fill, or to a drain container or house drain at the end of treatment. The fifth valve may be normally open to the sorbent cartridge and normally closed to drain to conserve energy since flow to the sorbent cartridge is more common. A sixth valve may be a three-way valve located downstream from the sorbent cartridge. The sixth valve determines whether regenerated PD fluid from the sorbent cartridge is reintroduced into the fresh PD fluid side of the PD machine or cycler or is alternatively, and at selected times, pumped through an ammonia sensor to confirm that the PD fluid has been properly cleaned and regenerated by the sorbent cartridge. The sixth valve may be normally open to the fresh PD fluid side of the PD machine or cycler to conserve energy since regenerated PD fluid flow bypassing the ammonia sensor is more common.

A seventh valve may be a three-way concentrate valve. The seventh valve determines whether an electrolyte concentrate or a glucose concentrate is pumped into the fresh PD fluid side of the PD machine or cycler. The seventh valve may be normally open to whichever concentrate is used in greater volume to conserve energy.

In addition to the ammonia sensor, the PD system of the present disclosure may also include flow sensors, e.g., a flow sensor in each of the fresh and used PD fluid sides of the machine or cycler. An inline PD fluid heater and associated one or more temperature sensor may be provided along the fresh PD fluid side of the machine or cycler for heating the fresh PD fluid to body temperature or 37° C. One or more conductivity sensor may be provided, e.g., one in each of the fresh and used PD fluid sides of the machine or cycler. Pressure sensors may also be provided, for example, one in each of the fresh and used PD fluid sides of the machine or cycler, and a third pressure sensor just upstream of the sorbent cartridge.

The machine or cycler in an embodiment includes a control unit that controls the pumps and valves and that receives outputs from each of the sensors discussed herein, which may be used as feedback for various purposes. For example, the control unit uses the output of the ammonia sensor to ensure that the sorbent cartridge is properly removing urea and other toxins from the used PD fluid. The control unit uses the outputs from the fresh and used flow sensors to set fresh and used PD fluid flowrates. The flowrate outputs may also be integrated over time to yield (i) how much fresh PD fluid has been delivered to the patient, (ii) how much used PD fluid has been removed from the patient, and (iii) a difference between (ii) versus (i) to know how much ultrafiltration (“UF”) or excess water has been removed from the patient. The control unit uses the output from the one or more temperature sensor for feedback in controlling the inline PD fluid heater, e.g., via a proportional, integral, derivative (“PID”) control algorithm.

The control unit uses the output of the fresh PD fluid pressure sensor as feedback to ensure that the positive pressure of fresh PD fluid delivered to the patient is within a positive patient pressure limit (e.g., 3.0 psig (0.21 bar) or less). The control unit uses the output of the used PD fluid pressure sensor as feedback to ensure that the negative pressure of used PD fluid removed from the patient is within a negative patient pressure limit (e.g., at or between −1.5 psig (−0.10 bar) and zero psig).

The control unit may use the output from the fresh PD fluid conductivity sensor to ensure that the electrolyte and glucose concentrates have been added in a proper amount to the regenerated PD fluid from the sorbent cartridge. The control unit may use the output from the used PD fluid conductivity sensor may interrogate the used dialysis fluid or patient effluent to look for solute removal in the patient's effluent (e.g., for urea, β₂ microglobulin, and/or creatinine) and/or for signs of peritonitis.

The PD system of the present disclosure also includes a number of passive devices or components, that is, components that are not under control of the control unit and that do not output to the control unit. The sorbent cartridge is one such passive component, which is used to clean and regenerate used PD fluid. Another passive component is the degassing tank, which receives the regenerated PD fluid from the sorbent cartridge and allows the regenerated PD fluid to pool for a period of time, where air degasses from the regenerated PD fluid via buoyancy and/or negative pressure applied to the degassing tank. The degassing tank also provides an area for the electrolyte and glucose concentrates to mix with the regenerated PD fluid. The degassing tank may include a first vent line leading to the air or vacuum pump and a second vent line leading to atmosphere. The second vent line may be capped with a hydrophobic vent that filters any air pulled into the degassing tank via the second vent line.

Yet another passive component supplied in an embodiment by the PD system of the present disclosure is a final or sterile stage microbial filter provided along the fresh PD fluid line to filter the regenerated PD fluid from the sorbent cartridge. The final or sterile stage filter may be a semi-reusable filter, such as an ultrafilter, which may be replaced every few months or so. The final or sterile stage filter provides a final sterilization step for the regenerated PD fluid, which serves as a check against any pathogens that may remain after sorbent cleaning and after the disinfection sequence discussed below.

Still another passive component supplied in an embodiment by the PD system of the present disclosure is a dialyzer located in the flexible line leading from the PD machine or cycler to the patient. The dialyzer helps to remove proteins from the used PD fluid or effluent before reaching the sorbent cartridge. The dialyzer also helps to remove large particles (e.g., fibrin) from the used PD fluid or effluent before reaching the sorbent cartridge. The dialyzer and flexible line leading from the PD machine or cycler to the patient are single use disposable components in one embodiment.

The control unit performs a plurality of treatment sequences using the above-described pumps, valves and sensors. The control unit performs a filling sequence by pumping new, fresh PD fluid to the patient, e.g., from a container or bag of fresh PD fluid through the final or sterile stage filter. The control unit may then perform a used dialysis fluid regeneration sequence by pumping used PD fluid from the patient, through the sorbent cartridge, to the degassing tank. In cooperation with the regeneration sequence, the control unit may perform concentrate dose sequences that dose the electrolyte and glucose concentrates into the degassing tank for mixing with and fortifying the regenerated PD fluid. The concentrates may be dosed in any desired order, e.g., electrolyte first, then glucose. The mixing and dosing in the degassing tank may lead to the generation of air. The control unit may therefore run a degassing sequence to pull air from the degassing tank and deliver the air to a drain container or house drain. At some point, e.g., prior to delivering the regenerated PD fluid to the patient, the control unit may run an ammonia checking sequence that pumps regenerated PD fluid past an ammonia sensor to confirm that the sorbent cartridge is operating properly. At the end of treatment, if the PD fluid is not to be left within the patient, e.g., for a last fill, the control unit in a drain sequence pumps used PD fluid from the patient to the drain container or house drain instead of through the sorbent cartridge to the degassing tank.

In a second main feature of the present disclosure, the PD system and associated methodology provides much of the fluid lines and components as reusable lines and components, e.g., having no disposable parts, which lowers disposable cost, waste and handling. The reusable lines and components are then disinfected after treatment, e.g., chemical and/or heat disinfected. The reusable components may include all pumps, valves and sensors. Any of the pumps discussed herein may be peristaltic, piston, gear, membrane or centrifugal pumps and have reusable components that contact fresh and used PD fluid over many treatments. The valves likewise have reusable components that contact fresh and used PD fluid over many treatments. The final or sterile stage filter as discussed herein may be reused for multiple treatments and then replaced periodically. All internal fluid lines are also reusable in one embodiment.

In one disinfection embodiment, the sources of PD fluid concentrate are removed after treatment and are replaced by a source of purified water (e.g., online or bagged), while the flexible patient line (e.g., including the dialyzer) is removed and a source of disinfectant, e.g., citric acid, is connected instead. In a disinfection sequence, the control unit causes any one or more of the pumps circulates citric acid disinfectant, which may be concentrated and combined with purified water, around a closed disinfection circuit or pathway, perhaps in multiple directions, while the heater may also heat the disinfectant to a desired temperature, e.g., 70° C. to 90° C., and while any one or more of the valves is toggled so that each line and component is sufficiently contacted. After the disinfection sequence, the control unit may run a final rinse sequence that flushes the disinfectant to drain using a purified water from the source of purified water.

In light of the disclosure set forth herein, and without limiting the disclosure in any way, in a first aspect of the present disclosure, which may be combined with any other aspect or portion thereof, a peritoneal dialysis (“PD”) system includes a fresh/regenerated PD fluid pump; a used PD fluid pump; a sorbent cartridge located fluidically between the fresh/regenerated PD fluid pump and the used PD fluid pump; a degassing tank located fluidically downstream from the sorbent cartridge; at least one valve located fluidically upstream of the degassing tank; and a control unit configured to control the fresh/regenerated PD fluid pump and the used PD fluid pump, the control unit further figured to operate the at least one valve to select whether fresh PD fluid or regenerated PD fluid is pumped by the fresh/regenerated PD fluid pump to the degassing tank.

In a second aspect of the present disclosure, which may be combined with any other aspect or portion thereof, the degassing tank is located fluidically upstream of the fresh/regenerated PD fluid pump.

In a third aspect of the present disclosure, which may be combined with any other aspect or portion thereof, the at least one valve includes a three-way-valve in fluid communication with the degassing tank, the sorbent cartridge and a fresh PD fluid container.

In a fourth aspect of the present disclosure, which may be combined with any other aspect or portion thereof, the at least one valve is a first at least one valve, and which includes a second at least one valve located fluidically between the first at least one valve and the degassing tank, the control unit further figured to operate the second at least one valve in a first state during treatment and in a second state during disinfection.

In a fifth aspect of the present disclosure, which may be combined with any other aspect or portion thereof, the at least one valve is a first at least one valve, and which includes a second at least one valve, the control unit further figured to operate the second at least one valve to select which of a plurality of concentrates is pumped to the degassing tank.

In a sixth aspect of the present disclosure, which may be combined with any other aspect or portion thereof, the PD system includes at least one of an electrolyte concentrate or a glucose concentrate in fluid communication with the second at least one valve.

In a seventh aspect of the present disclosure, which may be combined with any other aspect or portion thereof, the PD system includes a concentrate pump located fluidically between the second at least one valve and the degassing tank for pumping the selected concentrate.

In an eighth aspect of the present disclosure, which may be combined with any other aspect or portion thereof, the at least one valve is a first at least one valve, and which includes a second at least one valve located fluidically between the first at least one valve and the sorbent cartridge, the control unit further figured to operate the second at least one valve to select whether or not regenerated PD fluid is pumped through a PD fluid impurity detection sensor.

In a ninth aspect of the present disclosure, which may be combined with any other aspect or portion thereof, the PD fluid impurity detection sensor is an ammonia detection sensor.

In a tenth aspect of the present disclosure, which may be combined with any other aspect or portion thereof, the at least one valve is a first at least one valve, and which includes a second at least one valve located fluidically upstream of the sorbent cartridge, the control unit further figured to operate the second at least one valve to select whether used PD fluid is delivered to the sorbent cartridge or to a drain.

In an eleventh aspect of the present disclosure, which may be combined with any other aspect or portion thereof, the PD system includes a vent pump positioned to pump gas along a vent line from the degassing tank to a drain.

In a twelfth aspect of the present disclosure, which may be combined with any other aspect or portion thereof, the PD system includes at least one of a flow sensor, inline heater, temperature sensor, conductivity sensor or pressure sensor in fluid communication with the fresh/regenerated PD fluid pump.

In a thirteenth aspect of the present disclosure, which may be combined with any other aspect or portion thereof, the PD system includes at least one of a conductivity sensor, flow sensor or pressure sensor in fluid communication with the used PD fluid pump.

In a fourteenth aspect of the present disclosure, which may be combined with any other aspect or portion thereof, the pressure sensor is a first pressure sensor, and which includes a second pressure sensor located fluidically between the used PD fluid pump and the sorbent cartridge.

In a fifteenth aspect of the present disclosure, which may be combined with any other aspect or portion thereof, the PD system includes a conductivity sensor located fluidically between the sorbent cartridge and the at least one valve.

In a sixteenth aspect of the present disclosure, which may be combined with any other aspect or portion thereof, the PD system includes at least one of (i) a microbial filter located fluidically between the fresh/regenerated PD fluid pump and a flexible patient line or (ii) a dialyzer located along the flexible patient line.

In a seventeenth aspect of the present disclosure, which may be combined with any other aspect or portion thereof, the PD system includes (i) a patient fill valve and a patient drain valve in fluid communication with the flexible patient line or (ii) a single patient fill and drain valve in fluid communication with the flexible patient line.

In an eighteenth aspect of the present disclosure, which may be combined with any other aspect or portion thereof, the PD system is configured such that for disinfection the flexible patient line is replaced with (i) a disinfection line connected to a source of disinfectant or (ii) a purified water line connected to a source of purified water.

In a nineteenth aspect of the present disclosure, which may be combined with any other aspect or portion thereof, the PD system is configured such that for disinfection a source of fresh PD fluid is replaced with (i) a source of disinfectant or (ii) a source of purified water.

In a twentieth aspect of the present disclosure, which may be combined with any other aspect or portion thereof, the peritoneal dialysis (“PD”) system includes a fresh/regenerated PD fluid pump; a used PD fluid pump; a sorbent cartridge located fluidically between the fresh/regenerated PD fluid pump and the used PD fluid pump; a microbial filter positioned to filter the fresh or regenerated PD fluid pumped from the fresh/regenerated PD fluid pump; and a dialyzer positioned and arranged to receive fresh and used PD fluid.

In a twenty-first aspect of the present disclosure, which may be combined with any other aspect or portion thereof, the dialyzer is located along a disposable patient line.

In a twenty-second aspect of the present disclosure, which may be combined with any other aspect or portion thereof, any of the features, functionality and alternatives described in connection with any one or more of FIGS. 1 to 3 may be combined with any of the features, functionality and alternatives described in connection with any other of FIGS. 1 to 3.

In light of the above aspects and disclosure herein, it is accordingly an advantage of the present disclosure to provide a relatively volumetrically accurate automated peritoneal dialysis (“PD”) machine or cycler.

It is another advantage of the present disclosure to provide a PD cycler that achieves relatively precise pressure control.

It is a further advantage of the present disclosure to provide a PD cycler that uses a sorbent to regenerate used PD fluid, reducing disposable waste, and reducing the amount of stored dialysis fluid volume, thereby reducing patient burden and lowering logistical costs, e.g., shipping and waste removal.

It is yet another advantage of the present disclosure to provide a PD cycler that uses a sorbent to regenerate used PD fluid, reducing the amount of premade fresh dialysis fluid needed for a treatment, thereby lowering treatment cost.

It is yet a further advantage of the present disclosure to provide a PD cycler that disinfects its components and internal lines between treatments, reducing disposable waste, reducing shipping and logistics, and reducing patient storage requirements.

It is still another advantage of the present disclosure to provide a relatively quiet PD cycler.

It is still a further advantage of the present disclosure to provide a portable PD cycler, which may be a wearable cycler.

Moreover, it is an advantage of the present disclosure to provide an APD cycler that disinfects components after treatment, further allowing disposable waste and cost to be reduced, e.g., by allowing a disposable heating component to be eliminated.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a flow schematic view of a sorbent-based peritoneal dialysis (“PD”) system of the present disclosure in a treatment configuration.

FIG. 2 is a flow schematic view of the PD system of FIG. 2 in a disinfection mode.

FIG. 3 is a cross-sectional view of one suitable sorbent cartridge of the present disclosure.

### DETAILED DESCRIPTION

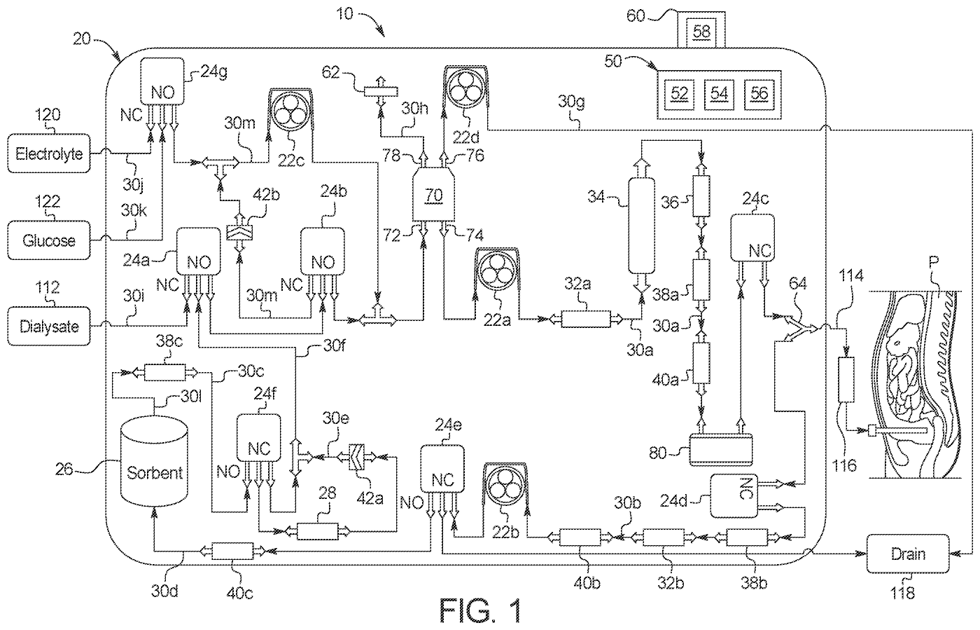

Referring now to the drawings and in particular to FIG. 1, an embodiment of an automated peritoneal dialysis (“APD”) system 10 and associated methodology of the present disclosure includes an APD machine or cycler 20, which is generally defined by the rectangular box in FIG. 1. In the illustrated embodiment, APD machine or cycler 20 includes a fresh/regenerated PD fluid pump 22a, a used PD fluid pump 22b, a concentrate pump 22c and a vent, air or vacuum pump 22d. Pumps 22a to 22d are illustrated as peristaltic pumps, however, pumps 22a to 22d may be any type of fluid pump, for example, a piston, gear, membrane or centrifugal pump, and may be of the same type or different types. Due to the reusable nature of system 10, pumps 22a to 22d are not limited to types that operate with a disposable item, such as a tube or a flexible chamber. Pumps 22a to 22d instead may include or define internal, e.g., metallic or partially metallic, cavities that receive and contact a fluid to be pumped, such as fresh or used dialysis fluid. On the other hand, pumps 22a to 22d may be peristaltic or membrane pumps that operate with a tube, flexible chamber, or other flexible fluid contacting portion that would in other circumstances be disposable, but which here are disinfected after treatment or prior to a subsequent treatment for reuse.

Cycler 20 of system 10 includes multiple valves, e.g., seven valves 24a to 24g. In an embodiment, multiple ones of the valves are three-way valves. The three-way valves are electrically actuated solenoid valves in one embodiment, each including a normally open (“NO”) port, a normally closed (“NC”) port and a common port. When energy is applied to the three-way valves, the ports switch states such that the NO port closes (restricts flow to the common port) and the NC port opens (allows flow to the common port). The NO port may be the more commonly used port to minimize the amount of time that the three-way valves need to be energized. The valves may alternatively or additionally include multiple two-way valves, which again may be electrically actuated solenoid valves that are energized open so as to be fail safe.

Due to the reuse of system 10, any of the valves described herein, including valves 24a to 24g, may include internal fluid contacting portions that are metallic or otherwise of a nature that would be cost prohibitive to discard after each treatment. In alternative embodiments, any of the valves described herein may operate with tubing (e.g., pinch valves) or flexible membranes (e.g., electric or pneumatic volcano valves), which are disinfected after treatment and reused. In still further alternative embodiments, any of the three-way valves described herein, including valves 24a, 24b and 24e to 24g, may be replaced via multiple two-way valves.

The valves include a first valve 24a, which is a three-way source valve that determines where PD fluid for a patient fill is sourced. Source valve 24a may be normally open to a sorbent cartridge 26 to receive regenerated PD fluid for a patient fill because more patient fills use regenerated PD fluid so less energy is used by making the NO port the regenerated port. Source valve 24a is normally closed to an initial, e.g., two-liter container or bag of fresh PD fluid 112. A second valve 24b may be a three-way valve located downstream from first valve 24a and be used primarily during patient fills for treatment (first state) and for creating a disinfection loop during a disinfection sequence (second state) at the end of treatment. Since the treatment state is more common than the disinfection state, second valve 24b is normally open to a treatment line extending between valves 24a and 24b.

A third valve 24c may be a two-way patient fill valve, which is normally closed to the patient for fail safe operation. Patient fill valve 24c is energized open during a patient fill in the illustrated embodiment. A fourth valve 24d may be a two-way patient drain valve, which is likewise normally closed to the patient for fail safe operation, and which is energized open during a patient drain. Providing separate fill and drain valves 24c and 24d allows fresh and used PD fluid to be separated and maintained in different permanent, reusable lines. In the illustrated embodiment, only a flexible patient line 114 leading from cycler 20 to a patient P receives both fresh and used PD fluid.

A fifth valve 24e may be a three-way valve located downstream from the two-way patient drain valve 24d. Fifth valve 24e determines where the used PD fluid from the patient is delivered, e.g., to sorbent cartridge 26 for regeneration for another patient fill, or to a drain container or house drain 118 at the end of treatment. Fifth valve 24e may be normally open to sorbent cartridge 26 and normally closed to drain 118 to conserve energy since flow to the sorbent cartridge is more common. A sixth valve 24f may be a three-way valve located downstream from sorbent cartridge 26. Sixth valve 24f determines whether regenerated PD fluid from sorbent cartridge 26 is reintroduced into the fresh PD fluid side of PD machine or cycler 20 or is alternatively, and at selected times, bypassed through an impurity detection sensor (e.g., ammonia sensor) 28 to confirm that the PD fluid has been properly cleaned and regenerated by the sorbent cartridge. Sixth valve 24f may be normally open to the fresh PD fluid side of the PD machine or cycler 20 to conserve energy since regenerated PD fluid more commonly does not flow through impurity detection or ammonia sensor 28.

A seventh valve 24g may be a three-way concentrate valve. Seventh valve determines whether an electrolyte concentrate 120 or a glucose concentrate 122 is pumped into the fresh PD fluid side of PD machine or cycler 20. Seventh valve 24g may be normally open to whichever concentrate is used in greater volume to conserve energy.

In addition to impurity detection sensor (e.g., ammonia sensor) 28, PD system 10 in the illustrated embodiment may also provide, along a fresh PD fluid line 30a and used PD fluid line 30b, respective flow sensors 32a, 32b, e.g., a flow sensor in each of the fresh and used PD fluid sides of machine or cycler 20. Flow sensors 32a, 32b may be inline, durable and in one example magnetic flow sensors. Other suitable invasive flow sensors include rotary vane, vortex shedding, optical, and mass flow sensors for example. Non-invasive flow sensors may also be provided and include heat pulse, time of flight and optical flow sensors, for example.

An inline PD fluid heater 34 and associated one or more temperature sensor 36 may be provided along fresh PD fluid line 30a of machine or cycler 20 for heating fresh PD fluid to body temperature or 37° C. Inline heater 34 is durable in one embodiment and is configured so as to be able to heat fresh or regenerated PD fluid from, e.g., 10° C. to body temperature or 37° C. over flowrates ranging from, e.g., 50 ml/min to 300 ml/min. Inline heater 34 may be a flow through and/or a circulation heater. Temperature sensor 36 may be a thermocouple or thermistor for example.

Machine or cycler 20 of system 10 in the illustrated embodiment includes conductivity sensors 38a to 38c, e.g., one each along fresh PD fluid line 30a (sensor 38a), used PD fluid line 30b (sensor 38b), and a sorbent outlet line 30c (sensor 38c) extending between sorbent cartridge 26 and three-way valve 24f. Conductivity sensors 38a to 38c may be inline, durable, and temperature compensated conductivity sensors, such as temperature compensated graphite probes.

Machine or cycler 20 of system 10 in the illustrated embodiment includes pressure sensors 40a to 40c, e.g., one each along fresh PD fluid line 30a (sensor 40a), used PD fluid line 30b (sensor 40b), and a sorbent inlet line 30d (sensor 40c) extending between three-way valve 24e and sorbent cartridge 26. PD fluid pressure sensor 40a to 40c may be durable, invasive, inline pressures sensor through which fresh, regenerated and used PD fluid flows. PD fluid pressure sensors 40a to 40c may alternatively be pod-type pressure sensors having a flexible diaphragm separating a fluid contacting side from an air side leading to a pressure transducer. Further alternatively, PD fluid pressure sensors 40a to 40c may be force sensors that abut directly against flexible portions of fresh PD fluid line 30a, used PD fluid line 30b and sorbent inlet line 30d, respectively. Still further alternatively, fresh and used PD fluid pressure sensors 40a and 40b may be combined into a force single sensor that abuts directly against flexible patient line 114, which receives both fresh and used PD fluid.

Impurity detection sensor (e.g., ammonia sensor) 28 is located along a regenerated PD fluid bypass line 30e extending from valve 24f back to a line 30f leading from valve 24f to valve 24a. A durable one-way or check valve 42a, such as a duck-billed check valve, may be located along regenerated PD fluid bypass line 30e to prevent regenerated PD fluid from backflowing through impurity detection sensor 28. Any one or more or all of the lines of cycler 20, including lines 30a to 30??? discussed herein may be durable, reusable and be made of a medically fluid safe metal, such as stainless steel, or of a suitable plastic, which are in one embodiment biocompatible, heat-disinfectable, and chemical-disinfectable. Suitable plastics may include polyvinyl chloride (“PVC”), polyethylene (“PE”), polyurethane (“PU”) and/or polycarbonate (“PC”).

In the illustrated embodiment of cycler 20 of system 10, each of pumps 22a to 22d, valves 24a to 24g, and inline heater 34 are powered and controlled via a control unit 50, which includes one or more processor 52, one or more memory 54 and a video controller 56 for controlling a video monitor 58. Video monitor 58 is part of an overall user interface 60 for system 10. User interface 60 includes any one or more of a touch screen overlay operable with video monitor 58 and/or one or more electromechanical input device, e.g., membrane switches, for inputting information into control unit 50. Video monitor 58 and speakers (e.g., operable with a sound card of control unit 50) are provided to output information to the patient or user, e.g., alarms, alerts and/or voice guidance commands.

Similarly, each of flow sensors 32a, 32b, temperature sensor 36, conductivity sensors 38a to 38c, and pressure sensors 40a to 40c outputs to control unit 50. Control unit 50 uses the sensor outputs to control and monitor the components and their functions for system 10. Control unit 50 is also programmed to run each of the flow sequences for system 10) described herein. Control unit 50 may also include a transceiver and a wired or wireless connection to a network, e.g., the internet, for sending treatment data to and receiving prescription instructions from a doctor's or clinician's server interfacing with a doctor's or clinician's computer.

Control unit 50 uses the outputs from flow sensors 32a, 32b to know how much fresh or regenerated PD fluid has been pumped by fresh/regenerated PD fluid pump 22a and possibly concentrate pump 22c (sensor 32a) and how much used PD fluid has been pumped by used PD fluid pump 22b (sensor 32b). The outputs from fresh and used PD fluid flow sensors 32a, 32b are used to control the flowrates of fresh and used PD fluid pumps 22a, 22b, respectively, to pump at desired or specified flowrates, e.g., by varying the power or input pulse train delivered to fresh and used PD fluid pumps 22a, 22b as needed. The outputs from flow sensors 32a and 32b are also integrated over time to yield (i) how much fresh and regenerated dialysis fluid is delivered to patient P (sensor 32a), (ii) how much used dialysis fluid is removed from patient P (sensor 32b), and (iii) a difference between (ii) versus (i) to know how much ultrafiltration (“UF”) or excess water has been removed from the patient.

Control unit 50 causes inline heater 34 to heat fresh dialysis fluid from its starting temperature to body fluid temperature, e.g., 37° C., for comfortable delivery to patient P. The output from temperature sensor 36 located downstream from dialysis fluid heater 34 is used as feedback to control the amount of heating power supplied to the heater. The feedback allows a target PD fluid temperature to be reached without significant overshoot. If needed for system 10, an upstream temperature sensor (not illustrated) may be provided, e.g., between flow sensor 32a and heater 34, for additional feedback, e.g., if incoming fluid to heater 34 is colder than usual then power to the heater is increased. Heating the fresh or regenerated PD fluid tends to separate dissolved air from the dialysis fluid. It is accordingly contemplated to locate heater 34 and temperature sensor 36 upstream from a degassing unit 70 discussed herein.

Control unit 50 uses the output of conductivity sensor 38a as feedback to ensure that electrolyte concentrate 120 and glucose concentrate 122 have been dosed properly by concentrate pump 22c into the regenerated PD fluid. The conductivity setpoint used by control unit 50 may correspond to a known standard level of 1.36% glucose PD fluid or 2.27% glucose PD fluid in various embodiments, or to some optimized glucose level that a clinician has determined and approved for the patient. System 10 allows for such optimization to occur. Control unit 50 in an embodiment also uses the output from conductivity sensor 38a as a check before allowing the fresh or regenerated PD fluid to be delivered to patient P. If for some reason the output from conductivity sensor 38a does not confirm that the fresh or regenerated PD fluid is suitable for patient delivery, then control unit 50 may cause fill valve 24c to close and user interface 60 to alarm. Control unit 50 may use the output from used PD fluid conductivity sensor 38b to interrogate used dialysis fluid to look for solute removal in the patient's effluent (e.g., for urea, β₂ microglobulin, and/or creatinine) or for signs of peritonitis. Control unit 50 may use the output from used PD fluid conductivity sensor 38c to interrogate the PD fluid outputted from sorbent cartridge 26 to ensure that its conductivity is within a desired or acceptable range that can be corrected via the addition of electrolyte concentrate 120 and glucose concentrate 122 such that the regenerated PD fluid meets a prescribed PD fluid, at least within acceptable limits. The outputs from conductivity sensor 38a may be temperature compensated via the reading from temperature sensor 36. Additional temperature sensors (not illustrated) may be employed to compensate the outputs of conductivity sensors 38b and 38c.

Control unit 50 uses the output of fresh pressure sensor 40a as feedback to ensure that the positive pressure of fresh PD fluid delivered to patient P from fresh PD fluid pump 22a is within a positive patient pressure limit (e.g., 3.0 psig (0.21 bar) or less). Control unit 50 uses the output of used pressure sensor 40b as feedback to ensure that the negative pressure of used PD fluid removed from patient P via used PD fluid pump 22b is within a negative patient pressure limit (e.g., at or between −1.5 psig (−0.10 bar) and zero psig). Control unit 50 uses the output of sorbent pressure sensor 40c as feedback to ensure that the incoming positive pressure of used PD fluid into sorbent cartridge 26 is within an allowable limit, which is sorbent dependent, but which may be about 1 bar (14.5 psig), and to look for blockages in the inlet line 30d to the sorbent cartridge.

PD system 10 also includes a number of passive devices or components, that is, components that are not under control of the control unit 50 and that do not output to the control unit. Sorbent cartridge 26 is one such passive component, which is used to clean and regenerate used PD fluid. Another passive component is a degassing tank 70, which receives regenerated PD fluid from sorbent cartridge 26 (and fresh PD fluid from fresh PD fluid container or bag 112) and allows the regenerated PD fluid to pool for a period of time, where air, CO₂ and any other gas is allowed to be degassed from the regenerated PD fluid via buoyancy and/or negative pressure applied to the degassing tank. Degassing tank 70 also provides an area for electrolyte concentrate 120 and glucose concentrate 122 to mix with the regenerated PD fluid from sorbent cartridge 26 to form a final regenerated PD fluid having a composition prescribed for patient P (e.g., having a conductivity as measured by conductivity sensor 38a corresponding to a known standard level of PD fluid, e.g., 1.36% glucose PD fluid or 2.27% glucose). To this end, degassing tank 70 may include one or more temperature compensated conductivity sensor (e.g., sensor 38a) outputting to control unit 50 to confirm that electrolyte concentrate 120 and glucose concentrate 122 have been dosed properly to achieve a desired or prescribed composition.

Degassing tank 70 is durable and reusable and may be formed of any of the metals or polymers listed herein. Degassing tank 70 in the illustrated embodiment includes or forms a fluid inlet 72, which receives (i) fresh PD fluid from fresh PD fluid container or bag 112, (ii) regenerated PD fluid from sorbent cartridge 26, and in combination with the regenerated PD fluid (iii) electrolyte concentrate 120 and glucose concentrate 122 via concentrate pump 22c. Degassing tank 70 includes or forms a fluid outlet 74, from which fresh PD fluid pump 22a pulls fresh PD fluid or final regenerated PD fluid, homogeneously mixed with electrolyte concentrate 120 and glucose concentrate 122, and pushes same along fresh PD fluid line 30a for delivery to patient P.

Degassing tank 70 includes or forms a first vent outlet 76, from which vent, air or vacuum pump 22d pulls gases such as air and CO₂ and pushes same along vent line 30g to drain container or house drain 118. Vent, air or vacuum pump 22d creates a negative pressure within degassing tank 70 that helps to separate gases such as air and CO₂ from the PD fluid residing within the degassing tank. Degassing tank 70 also includes or forms a second vent outlet 78 leading to a second vent line 30h that opens to atmosphere. Second vent line 30h in the illustrated embodiment is capped with a hydrophobic membrane vent 62 that filters any air pulled into degassing tank 70 via second vent line 30h. Although not illustrated, a valve under control of control unit 50 may be placed along second vent line 30h to help maintain a negative pressure within degassing tank 70 (valve closed) or allow filtered air to enter the degassing tank (valve open).

Yet another passive component supplied in an embodiment by PD system 10 of the present disclosure is a final or sterile stage microbial filter 80 provided along fresh PD fluid line 30a to filter regenerated PD fluid received from sorbent cartridge 26. Final or sterile stage microbial filter 80 may be a semi-reusable filter, such as an ultrafilter, which may be replaced every few months or so. Final or sterile stage microbial filter 80 provides a final sterilization step for the regenerated PD fluid, which serves as a check against any pathogens that may remain after sorbent cleaning and after the disinfection sequence discussed below. That is, final or sterile stage microbial filter 30 is configured to filter the finally or partially mixed PD fluid into an injectable quality for delivery to patient P. Where sterile stage microbial filter 80 is a semi-reusable filter (e.g., ultrafilter), the filter is reusable for a number of uses or service hours after which it is replaced by a service person, patient P or caregiver depending on its location and connection to cycler 20. Here, filter 80 may for example be configured with self-sealing quick disconnect connectors that allow filter 80 to be easily plugged into and removed from a readily accessible surface or cavity of cycler 20.

Still another passive component supplied in an embodiment by the PD system of the present disclosure is a dialyzer 116 located along flexible patient line 114 leading from PD machine or cycler 20 to patient P. Dialyzer 116 helps to remove proteins from the used PD fluid or effluent before reaching sorbent cartridge 26. Dialyzer 116 also helps to remove large particles (e.g., fibrin) from the used PD fluid or effluent before reaching sorbent cartridge 26. Dialyzer 116 further serves as a final stage of filtration for the fresh or regenerated PD fluid delivered for a patient fill and may therefore eliminate the need for final or sterile stage microbial filter 80. Flow is from top to bottom through the two illustrated ports of dialyzer 116 for a patient fill, and from bottom to top through the two ports of dialyzer 116 for a patient drain. Dialyzer 116 and flexible patient line 114 leading from PD machine or cycler 20 to patient P are single use disposable components in one embodiment.

Control unit 50 performs a plurality of treatment sequences using the above-described pumps, valves, sensors and passive components of system 10. Referring still to FIG. 1, control unit 50 performs an initial filling sequence with fresh PD fluid by energizing three-way valve 24a, opening patient fill valve 24c and pumping via fresh PD fluid pump 22a new; fresh PD fluid to patient P, e.g., from fresh PD fluid container or bag 112, through valves 24a and 24b, through degassing tank 70, past sensors 32a, 36, 38a and 40a, through inline heater 34 to be heated to body temperature or 37° C., through sterile stage microbial filter 80, patient fill valve 24c and dialyzer 116 to patient P. Pressure sensor 40a provides feedback to control unit 50 for controlling positive pumping pressure as discussed herein. Control unit 50 monitors and integrates the output of flow sensor 32a so that when a prescribed fill volume has been delivered to patient P, three-way valve 24a is de-energized, fresh PD fluid pump 22a is stopped, and the initial patient fill using fresh PD fluid from fresh PD fluid from container or bag 112 is complete. Due to the dialysis fluid regeneration discussed herein, container or bag 112 may be sized to hold only a single patient fill volume, e.g., around two liters.

After the patient fill sequence, control unit allows the fresh PD fluid to dwell within the peritoneal cavity of patient P for a prescribed amount of time, e.g., on the order of an hour, to remove waste and toxins from the patient into the indwelling PD fluid.

Referring still to FIG. 1, control unit 50 after the patient dwell performs a used dialysis fluid regeneration sequence by opening patient drain valve 24d and pumping via used PD fluid pump 22b used PD fluid from patient P and through dialyzer 116, patient drain valve 24d, past sensors 38b, 32b, 40b, though three-way valve 24e, past sorbent pressure sensor 40c, through sorbent cartridge 26, past sorbent conductivity sensor 38c, through three-way valves 24f, 24a and 24b, into degassing tank 70. Pressure sensor 40b provides feedback to control unit 50 for controlling negative pumping pressure as discussed herein. Pressure sensor 40c in addition to assuring safe pressure to sorbent cartridge 26 is also able to detect blocks due to effluent fibrin entering the chambers of sorbent cartridge 26 (effluent fibrin should be removed by and large via dialyzer 116). The pressure checking also helps to avoid leaks in lines 30b and 30d leading to sorbent cartridge 26.

In a first regeneration sequence following a first patient fill, sorbent cartridge 26 is primed, wherein any air is pushed to degassing tank 70. Introducing the used PD fluid into the bottom of sorbent cartridge 26 and flowing the used PD fluid up against gravity ensures that sorbent cartridge 26 priming and toxin removal from the used PD fluid is performed efficiently. The sorbent chemicals (e.g., layers of activated carbon, urease, zirconium phosphate, ZoC, etc.) absorb the toxins from the used PD fluid. Control unit 50 uses the output of conductivity sensor 38c to confirm that PD fluid exiting sorbent cartridge 26 has been properly regenerated. Degassing tank 70 allows all gases (air, CO2, etc.) to be removed from the regenerated PD fluid before returning to patient P.

Referring still to FIG. 1, in cooperation with the regeneration sequence just described, control unit 50 may perform concentrate dose sequences that dose electrolyte concentrate 120 and glucose concentrate 122 into degassing tank 70 for mixing with and refortifying the regenerated PD fluid to form a final regenerated PD fluid suitable for delivery to patient P. Control unit 50 runs concentrate pump 22c and toggles three-way concentrate valve 24g to dose the concentrates in any desired order, e.g., electrolyte 120 first, then glucose 122. Control unit 50 in an embodiment coordinates the speed of concentrate pump 22c with the speed of fresh PD fluid pump 22a to dose electrolyte 120 and glucose 122 in a desired amount or concentration, which is confirmed via feedback from conductivity sensor 32a (or via one or more conductivity sensor located in degassing tank 70, which could be sensor 32a). Control unit 50 uses the conductivity feedback to control the speed of concentrate pump 22c relative to the speed of fresh PD fluid pump 22a to achieve a desired final regenerated PD fluid, e.g., having a known standard level of 1.36% glucose or 2.27% glucose. Customized glucose levels, e.g., between approved 1.36% and 2.27% glucose percentages may also be achieved.

Referring still to FIG. 1, the flow of regenerated PD fluid via used PD fluid pump 22b and the flow of concentrate via concentrate pump 22c cause turbulence and mixing within degassing tank 70, which may lead to the generation of air. Gases such as CO₂ are also produced in sorbent cartridge 26, which may be formed in a relatively large amount. Control unit 50 therefore runs a degassing sequence to pull air, CO₂ and any other gas from degassing tank 70 and deliver same to a drain container or house drain 118. In the illustrated embodiment, control unit 50 causes vent, air or vacuum pump 22d to apply a negative pressure within degassing tank 70 to pull gas microbubbles from the PD fluid, which may otherwise not release readily from solution via buoyancy alone. Vent, air or vacuum pump 22d also pushes the removed gases to drain 118.

In an embodiment, degassing tank 70 is sized to hold a fill volume's worth of regenerated PD fluid, e.g., around two liters. Once the prescribed fill volume of regenerated PD fluid is delivered to degassing tank 70, e.g., which is known via the integrating of the output from used PD fluid flow sensor 32b, any remaining effluent within patient P is delivered to drain by energizing drain valve 24e to open the normally closed port to drain 118 and continuing to run used PD fluid pump 22b. In various embodiment, control unit 50 of system 10 is programmed to end a patient drain when (i) a prescribed amount of used dialysis fluid (e.g., a factor such as 1.3 multiplied by the prescribed fill volume) has been removed from patient P or (ii) a characteristic signal or output change from pressure sensor 40b, e.g., characteristic negative pressure increase, is seen at control unit 50, which indicates that the patient is empty or virtually empty.

At some point during the filling of degassing tank 70 with the next fill sequence worth of regenerated PD fluid, control unit 50 runs an ammonia leakage checking sequence in which three-way valve 24f is energized to open the normally closed port to regenerated PD fluid bypass line 30e and impurity detection sensor (e.g., ammonia sensor) 28. The output of impurity detection sensor 28 is analyzed by control unit 50 to confirm that sorbent cartridge 26 is operating properly to remove toxins from the used PD fluid. Impurity detection or ammonia sensor 28 may be an optical sensor that reads a color change of the regenerated PD fluid sample to measure its total ammonia and ammonium concentration. Control unit 50 may also use the output of impurity detection or ammonia sensor 28 to monitor the effectiveness of sorbent cartridge 26, e.g., to predict when the sorbent cartridge may no longer be effective. If impurity detection or ammonia sensor 28 detects that sorbent cartridge 26 is no longer effective, control unit 50 ends treatment and pumps any remaining used or regenerated PD fluid from patient P and degassing tank 70 to drain 118.

At the end of treatment, if the indwelling PD fluid is not to be left within the patient, e.g., for a last fill, control unit 50 in a drain sequence energizes drain valve 24e to open the normally closed port to the drain line and causes used PD fluid pump 22b to pump used PD fluid from the patient to drain container or house drain 118 instead of through sorbent cartridge 26 to degassing tank 70. Control unit 50 ends the final drain sequence via any of the methods discussed herein. As discussed earlier, control unit 50 may integrate the total amount of fresh and regenerated PD fluid delivered to patient P over all patient fills via the output from flow sensor 32a and subtract that total fill amount from a total drain amount formed by integrating the total amount of used PD fluid removed from patient P over all patient drains via the output from flow sensor 32b to arrive at a total amount of ultrafiltration removed from patient P over the course of a treatment. Additionally, the output of used PD fluid conductivity sensor 38b may be used to evaluate treatment effectiveness, and/or look for signs of peritonitis, for example.

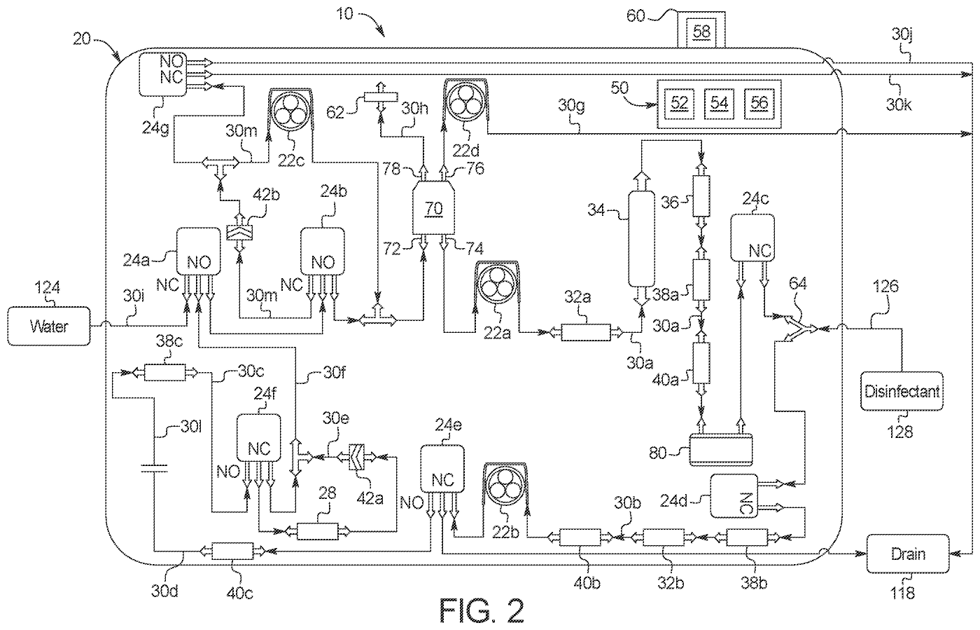

Referring now to FIG. 2, at the end of treatment and after patient P has disconnected from flexible patient line 114, the patient or caregiver (in any order) removes flexible patient line 114 and associated dialyzer 116 from machine or cycler 20, removes fresh PD fluid container or bag 112 from solution line 30i, removes electrolyte container 120 from electrolyte line 30j, removes glucose container 122 from glucose line 30k, and removes sorbent cartridge 26 from sorbent inlet line 30d and sorbent outlet line 30l. The patient or caregiver then (in any order) attaches solution line 30i to a source of purified water 124 (bagged or online), attaches or runs electrolyte line 30j and glucose line 30k to drain container or house drain 118, attaches a disinfectant line 126 and disinfectant container 128 to a durable patient Y- or T-connector 64 located at the housing of machine or cycler 20, and connects the ends of sorbent inlet line 30d and sorbent outlet line 301 together to form a closed disinfection loop. The disinfectant used herein may be any suitable disinfectant, such as citric acid.

In a disinfection sequence, control unit 50 causes patient drain valve 24d to open and used PD fluid pump 22b to pull disinfectant past all sensors located along used PD fluid line 30b and sensor 38c in sorbent outlet line 301. Three-way valve 24f is toggled to flow disinfectant past impurity detection sensor (e.g., ammonia sensor) 28. The used PD fluid side of machine or cycler 20 is thereby primed with disinfectant. Fresh PD fluid pump 22a then pulls additional disinfectant into the fresh PD fluid side of machine or cycler 20 to reach all sensors, inline heater 34 and final or sterile stage microbial filter 80 located on the fresh PD fluid side of machine or cycler 20. Valve 24b is energized to allow disinfectant to be pumped through a disinfection line 30m via concentrate pump 22c to reach all lines downstream of the concentrate pump. A second one-way or check valve 42b, such as a duck-billed check valve, is located along disinfection line 30m to allow concentrate pump 22c to be reversed to push disinfectant through concentrate lines 30j and 30k illustrated in FIG. 2.

Once machine or cycler 20 is fully primed with disinfectant, control unit 50 actuates all pumps with inline heater 34 energized to heat the disinfectant to a desired disinfection temperature, e.g., 70° C. to 90° C., and during which any one or more or all of the valves are toggled so that each line and component of machine or cycler 20 is sufficiently contacted with heated disinfectant. Once all sensors, lines, valves, pumps and filter 80 have received a desired disinfection dose, e.g., desired A₀ disinfection dose, control unit 50 ends the disinfection sequence.

Control unit 50 then runs fresh PD fluid pump 22a and concentrate pump 22c in forward and/or reverse directions with valve 24a energized to open its normally closed port, while toggling valves 24c and 24g, to allow purified water to rinse disinfectant from the fresh PD fluid side of machine or cycler 20 to drain 118. Control unit 50 then runs used PD fluid pump 22b in forward and/or reverse directions with valve 24a energized to open its normally closed port, while toggling valves 24d and 24f, to allow purified water to rinse disinfectant from the used PD fluid side of machine or cycler 20 to drain 118.

It should be appreciated that the locations of purified water source 124 and disinfectant source 128 could be reversed to run the disinfection and purified water rinse sequences just described with inline heater 34 heating the disinfectant to a desire disinfection temperature during the disinfection sequence. The pumping and valving sequences are modified as needed. It should also be appreciated that system 10 provides much of the fluid lines and components as reusable lines and components, e.g., having no disposable parts, which lowers disposable cost, waste and handling. The reusable lines and components are disinfected after treatment, e.g., chemical and/or heat disinfected as described above. The reusable components may include all pumps, valves and sensors. Any of the pumps discussed herein may be peristaltic, piston, gear, membrane or centrifugal pumps and have reusable components that contact fresh and used PD fluid over many treatments. The valves likewise have reusable components that contact fresh and used PD fluid over many treatments. The final or sterile stage filter as discussed herein may be reused for multiple treatments and then replaced periodically. All internal fluid lines are also reusable in one embodiment.

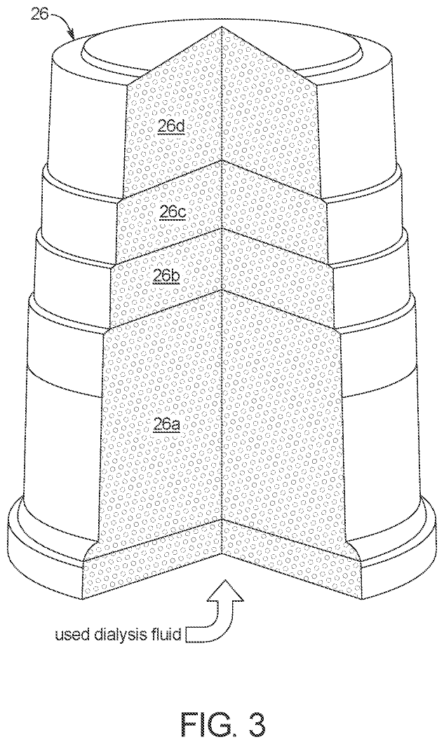

Referring now to FIG. 3, one suitable embodiment for sorbent cartridge 26 is illustrated. In the illustrated embodiment, sorbent cartridge 26 includes a bottom activated carbon layer 26a that binds heavy metals, oxidants, chloramines, creatinine, uric acid, other organics and middle molecules. A second layer 26b includes urease that converts urea and releases ammonium carbide. A third layer 26c includes zirconium phosphate that binds ammonium, calcium, magnesium, potassium, metals and other cations and releases sodium and hydrogen. A fourth layer 26d includes zirconium oxide and zirconium carbonate that binds phosphate, fluoride and heavy metals and releases acetate, bicarbonate and sodium.

It should be understood that various changes and modifications to the presently preferred embodiments described herein will be apparent to those skilled in the art. For example, while the fluid schematics illustrated herein show connections to specific NO and NC ports of valves 24a, 24b and 24e to 24g forming one workable overall flow schematic, the present disclosure is not limited to the specific NO and NC connections, and those of skill may determine others. Also, while a combined chemical and heat disinfection is disclosed, chemical or heat alone may be sufficient. Other types of disinfection, e.g., ultraviolet light, may be used additionally or alternatively. Moreover, while flow sensing for flowrate and integration for volume control is disclosed for cycler 20, inherently accurate pumps, such as piston pumps, or volumetric control components, such as balance chambers, may be used instead. Still further, the order and location of the sensors described herein may be varied. Further additionally, while separate fill and drain valves 24c and 24d are disclosed, a single fill/drain valve may be provided alternatively, wherein fresh/regenerated PD fluid pump 22a and used PD fluid pump 22b also operate as clamps (e.g., peristaltic clamping) to allow fresh or used PD fluid to flow through the single fill/drain valve. It is therefore intended that any or all of such changes and modifications may be covered by the appended claims.

The invention is claimed as follows:

1. A peritoneal dialysis (“PD”) system comprising:
a fresh/regenerated PD fluid pump;
a used PD fluid pump;
a sorbent cartridge located fluidically between the fresh/regenerated PD fluid pump and the used PD fluid pump;
a degassing tank located fluidically downstream from the sorbent cartridge;
at least one valve located fluidically upstream of the degassing tank; and
a control unit configured to control the fresh/regenerated PD fluid pump and the used PD fluid pump, the control unit further figured to operate the at least one valve to select whether fresh PD fluid or regenerated PD fluid is pumped by the fresh/regenerated PD fluid pump to the degassing tank.

2. The PD system of claim 1, wherein the degassing tank is located fluidically upstream of the fresh/regenerated PD fluid pump.

3. The PD system of claim 1, wherein the at least one valve includes a three-way-valve in fluid communication with the degassing tank, the sorbent cartridge and a fresh PD fluid container.

4. The PD system of claim 1, wherein the at least one valve is a first at least one valve, and which includes a second at least one valve located fluidically between the first at least one valve and the degassing tank, the control unit further figured to operate the second at least one valve in a first state during treatment and in a second state during disinfection.

5. The PD system of claim 1, wherein the at least one valve is a first at least one valve, and which includes a second at least one valve, the control unit further figured to operate the second at least one valve to select which of a plurality of concentrates is pumped to the degassing tank.

6. The PD system of claim 5, which includes at least one of an electrolyte concentrate or a glucose concentrate in fluid communication with the second at least one valve.

7. The PD system of claim 5, which includes a concentrate pump located fluidically between the second at least one valve and the degassing tank for pumping the selected concentrate.

8. The PD system of claim 1, wherein the at least one valve is a first at least one valve, and which includes a second at least one valve located fluidically between the first at least one valve and the sorbent cartridge, the control unit further figured to operate the second at least one valve to select whether or not regenerated PD fluid is pumped through a PD fluid impurity detection sensor.

9. The PD system of claim 8, wherein the PD fluid impurity detection sensor is an ammonia detection sensor.

10. The PD system of claim 1, wherein the at least one valve is a first at least one valve, and which includes a second at least one valve located fluidically upstream of the sorbent cartridge, the control unit further figured to operate the second at least one valve to select whether used PD fluid is delivered to the sorbent cartridge or to a drain.

11. The PD system of claim 1, which includes a vent pump positioned to pump gas along a vent line from the degassing tank to a drain.

12. The PD system of claim 1, which includes at least one of a flow sensor, inline heater, temperature sensor, conductivity sensor or pressure sensor in fluid communication with the fresh/regenerated PD fluid pump.

13. The PD system of claim 1, which includes at least one of a conductivity sensor, flow sensor or pressure sensor in fluid communication with the used PD fluid pump.

14. The PD system of claim 13, wherein the pressure sensor is a first pressure sensor, and which includes a second pressure sensor located fluidically between the used PD fluid pump and the sorbent cartridge.

15. The PD system of claim 1, which includes a conductivity sensor located fluidically between the sorbent cartridge and the at least one valve.

16. The PD system of claim 1, which includes at least one of (i) a microbial filter located fluidically between the fresh/regenerated PD fluid pump and a flexible patient line or (ii) a dialyzer located along the flexible patient line.

17. The PD system of claim 16, which includes (i) a patient fill valve and a patient drain valve in fluid communication with the flexible patient line or (ii) a single patient fill and drain valve in fluid communication with the flexible patient line.

18. The PD system of claim 16, which is configured such that for disinfection the flexible patient line is replaced with (i) a disinfection line connected to a source of disinfectant or (ii) a purified water line connected to a source of purified water.

19. The PD system of claim 1, which is configured such that for disinfection a source of fresh PD fluid is replaced with (i) a source of disinfectant or (ii) a source of purified water.

20. A peritoneal dialysis (“PD”) system comprising:
a fresh/regenerated PD fluid pump;
a used PD fluid pump;
a sorbent cartridge located fluidically between the fresh/regenerated PD fluid pump and the used PD fluid pump;
a microbial filter positioned to filter the fresh or regenerated PD fluid pumped from the fresh/regenerated PD fluid pump; and
a dialyzer positioned and arranged to receive fresh and used PD fluid.

21. A peritoneal dialysis (“PD”) system of claim 20, wherein the dialyzer is located along a disposable patient line.
